(19) United States
(12) Patent Application Publication
(10) Pub. No.: US 2008/0261330 A1
(43) Pub. Date: Oct. 23, 2008

(54) Apparatus Components and Methods of Using Apparatus Components to Detect the Presence of an Analyte

(76) Inventors: Patrick Brems, Worms (DE); Gonda Van Essche, Worms (DE); Maria Martha Rhemrev-Boom, Nijeveen (NL)

Correspondence Address: William D Bunch;W R Grace & Co Conn Patent department legal, 7500 Grace Drive, Columbia, MD, 21044, US

(21) Appl. No.: 11/660,125

(22) PCT Filed: Jul. 19, 2005
(86) PCT No.: PCT/EP2005/007873

§ 371 Date: May 12, 2008

Related U.S. Application Data
(60) Provisional application No. 60/589,074, filed on Jul. 19, 2004.

(51) Int. Cl. G01N 33/544; B01J 19/00

(52) U.S. Cl. 436/528; 422/70; 422/99

(57) ABSTRACT

Apparatus components including rigid supports suitable for use in an affinity column, affinity columns, and an affinity columns in fluid communication with an analytical column, such as in a high pressure liquid chromatography (HPLC) column, are disclosed. Methods of using the apparatus components to detect the presence of one or more analytes are also disclosed.

### FIELD OF THE INVENTION

The present invention relates generally to apparatus components including rigid supports suitable for use in affinity columns, affinity columns, and apparatus comprising an affinity column in fluid communication with an analytical column, such as a high pressure liquid chromatography (HPLC) column. The present invention further relates to methods of using the apparatus components to detect the presence of one or more analytes.

### BACKGROUND OF THE INVENTION

Apparatus components and methods for analyzing test samples that potentially contain one or more analytes are known. However, there exists a need in the art of sample analysis for one or more of the following benefits:

(1) apparatus components used alone or in combination with one another that enable less sample preparation steps and less sample handling steps;

(2) methods that enable less sample preparation steps and less sample handling steps;

(3) apparatus components used alone or in combination with one another that enable highly precise analysis of complex test samples for a given analyte with minimal interference from (i) non-analyte components within the complex test sample (i.e., undesirable bonding of materials other than the target analyte to one or more ligands used in the apparatus), and (ii) undesirable bonding of the target analyte to reactive sites other than ligands used in the apparatus;

(4) apparatus components used alone or in combination with one another that enable highly precise analysis of complex test samples for specific analytes (e.g., analytes having estrogenic activity) with minimal interference from (i) non-analyte components within the complex test sample (i.e., undesirable bonding of materials other than the target analyte to one or more ligands used in the apparatus), and (ii) undesirable bonding of the target analyte to reactive sites other than ligands used in the apparatus; and

(5) the ability to utilize an affinity column on-line or in fluid communication with an analytical column.

### SUMMARY OF THE INVENTION

The present invention is directed to apparatus components including rigid supports suitable for use in affinity columns, affinity columns containing rigid supports, and apparatus containing an affinity column in fluid communication with an analytical column, such as a high pressure liquid chromatography (HPLC) column. The apparatus components may be used to capture and quantify one or more analytes from a variety of complex mixtures.

In one embodiment of the present invention, the apparatus component comprises rigid supports suitable for use in affinity columns. One exemplary rigid support of the present invention comprises a plurality of inorganic particles, wherein each particle comprises (i) an inorganic substrate; (ii) a modified substrate surface that reduces non-specific binding of non-analyte materials (i.e., non-specific binding of materials other than the target analyte) and ligand-specific analyte materials (i.e., non-specific binding of the target analyte to reactive sites other than reactive sites provided by one or more ligands) to the inorganic substrate; and (iii) one or more ligands bonded to the inorganic substrate, wherein the one or more ligands comprises a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof. In exemplary embodiments of the present invention, the inorganic particles comprise inorganic metal oxide particles, such as silica or silica gel particles.

The present invention is further directed to affinity columns containing a rigid support material. In an exemplary embodiment of the present invention, the affinity column comprises a column structure having a column volume; and a rigid support positioned in the column volume of the column structure, wherein the rigid support comprises a plurality of inorganic particles, wherein each particle comprises (i) an inorganic substrate; (ii) a modified substrate surface that reduces non-specific binding of non-analyte materials and ligand-specific analyte materials to the inorganic substrate; and (iii) one or more ligands bonded to the inorganic substrate, wherein the one or more ligands comprises a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof.

The present invention is even further directed to an apparatus comprising an affinity column in fluid communication with an analytical column, wherein the affinity column contains a rigid support (i) capable of withstanding a column pressure of up to about 200 bar, and (ii) having one or more ligands bonded thereto, wherein the one or more ligands are capable of selectively bonding to one or more analytes within a given sample solution. In one exemplary embodiment, the affinity column of the apparatus contains rigid support materials of the present invention.

The present invention is also directed to methods of preparing rigid supports, immunoaffinity columns, and apparatus containing an immunoaffinity column, as well as methods of using the rigid supports, immunoaffinity columns, and apparatus to detect the presence of one or more analytes in a given sample. The methods of the present invention may be used to analyze a test sample that potentially contains at least one analyte.

In one exemplary embodiment of the present invention, the present invention is directed to methods of making rigid support materials comprising an inorganic substrate. In one exemplary method, the method comprises the following steps: (1) attaching R groups to at least a first portion of the surface of the inorganic substrate, wherein the R groups have a reactivity less than any functional groups on a surface of the inorganic substrate prior to the attaching step; (2) attaching one or more linkers to at least a second portion of the surface of the inorganic substrate, wherein the one or more linkers comprise an aldehyde functional group; and (3) selectively bonding one or more ligands to the one or more linkers

In one exemplary embodiment of the present invention, the present invention is directed to methods of analyzing test samples that potentially contain at least one analyte. In one exemplary embodiment, the method of analyzing a test sample that potentially contains at least one analyte comprises the step of (a) introducing a test sample into an affinity column containing a rigid support, wherein the rigid support comprises a plurality of inorganic particles, wherein each particle comprises (i) an inorganic substrate; (ii) a modified substrate surface that reduces non-specific binding of non-analyte materials and ligand-specific analyte materials to the inorganic substrate; and (iii) one or more ligands bonded to the inorganic substrate, wherein the one or more ligands comprises a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof.

The exemplary method of analyzing a test sample that potentially contains at least one analyte may further comprise the following steps: (a) allowing the test sample to come into contact with the rigid support and ligands thereon; (b) rinsing the rigid support to wash away any test sample components that do not bond to the ligands; (c) introducing an eluent solution into the affinity column so that the eluent solution comes into contact with one or more analytes bound to the ligands on the rigid support; (d) allowing the eluent solution to remain in contact with the rigid support for a period of time so as to form an eluent sample potentially containing one or more analytes; and (e) analyzing contents on the analytical column to determine a presence of one or more analytes in the test sample.

In a further exemplary embodiment, the present invention is directed to a method of analyzing a test sample that potentially contains at least one compound having estrogenic activity, wherein the method comprises the steps of introducing the test sample into an affinity column containing a rigid support having one or more ligands bonded thereto, wherein the one or more ligands are capable of selectively bonding to one or more compounds having estrogen activity.

The present invention is further directed to methods of analyzing an eluent sample, wherein the method comprises the steps of transferring an eluent sample from an affinity column to an analytical column, wherein the affinity column is in fluid communication with the analytical column, and analyzing contents of the analytical column to determine the presence of one or more analytes in the eluent sample. For example, the eluent sample may contain a mycotoxin, folic acid, vitamin B₁₂ (cyanocobalamine), or a combination thereof.

In one exemplary embodiment, the method of analyzing an eluent sample comprises analyzing an eluent sample potentially containing at least one mycotoxin, wherein the method comprises the steps of transferring the eluent sample from an affinity column to an analytical column, wherein the affinity column is in fluid communication with the analytical column, and analyzing contents of the analytical column to determine a presence of least one mycotoxin in the eluent sample.

In a further exemplary embodiment, the method of analyzing an eluent sample comprises analyzing an eluent sample potentially containing folic acid, vitamin B₁₂ (cyanocobalamine), or a combination thereof, wherein the method comprises the steps of transferring the eluent sample from an affinity column to an analytical column, wherein the affinity column is in fluid communication with the analytical column, and analyzing contents of the analytical column to determine a presence of folic acid, vitamin B₁₂ (cyanocobalamine), or both in the eluent sample.

These and other features and advantages of the present invention will become apparent after a review of the following detailed description of the disclosed embodiments and the appended claims.

### BRIEF DESCRIPTION OF THE FIGURES

The present invention is further described with reference to the appended figures, wherein:

FIG. 1 depicts a schematic view of an exemplary apparatus of the present invention;

FIG. 2 depicts an exemplary affinity column of the present invention;

FIG. 3 depicts another exemplary apparatus of the present invention showing fluid flow through the apparatus during loading of a sample into a sample loop;

FIG. 4 depicts the exemplary apparatus of FIG. 3 during injection of a sample into the affinity column;

FIG. 5 depicts the exemplary apparatus of FIG. 3 during sample elution from the affinity column; and

FIG. 6 depicts the exemplary apparatus of FIG. 3 during sample detection.

### DETAILED DESCRIPTION OF THE INVENTION

The present invention is directed to apparatus components including (i) rigid supports suitable for use in affinity columns, (ii) affinity columns containing rigid supports, (iii) apparatus containing a rigid support and/or an affinity column of the present invention in combination with an analytical column, such as a high pressure liquid chromatography (HPLC) column, and (iv) apparatus containing an affinity column in fluid communication with an analytical column, such as a high pressure liquid chromatography (HPLC) column. The present invention is further directed to methods of making one or more of the apparatus components, as well as methods of using one or more of the apparatus components to analyze test samples, including complex mixtures, which potentially contain one or more analytes. The present invention is even further directed to methods of using one or more of the apparatus components to capture and/or quantify one or more analytes from a variety of complex mixtures.

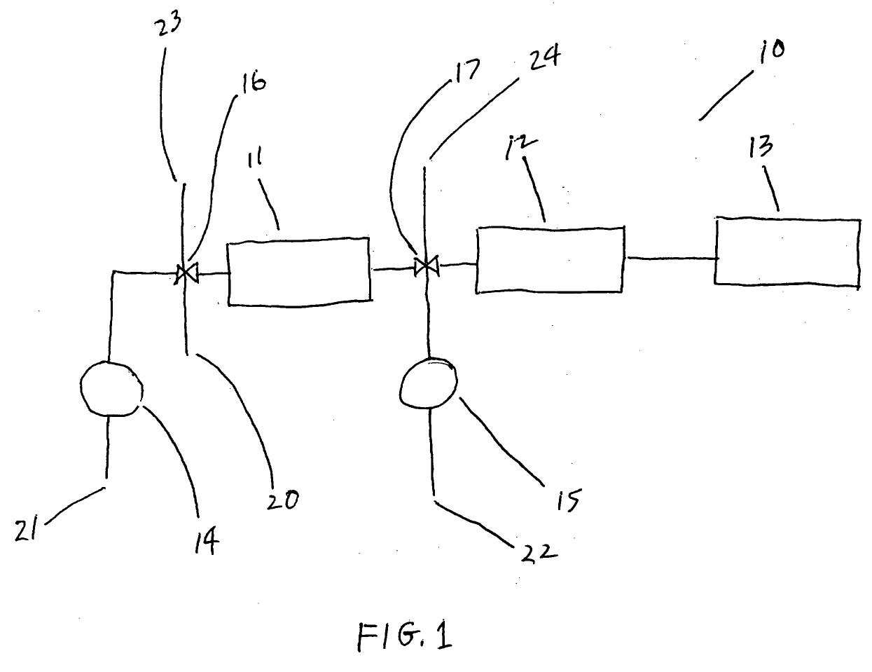

One exemplary apparatus 10 of the present invention is shown in FIG. 1. Exemplary apparatus 10 comprises affinity column 11, analytical column 12, detector 13, first pump 14, second pump 15, first valve 16, second valve 17, test sample inlet 20, first buffer inlet 21, elution buffer inlet 22, first waste outlet 23, and affinity column waste outlet 24. In one desired embodiment of the present invention, affinity column 11 and analytical column 12 are joined to one another via a coupling (not shown) so that affinity column 11 is in fluid communication with analytical column 12. As used herein, the term “in fluid communication with” describes an embodiment of the present invention wherein an eluent sample leaving an affinity column flows directly into an analytical column via a coupling between the affinity column and the analytical column. Such an arrangement (also referred to herein as an “on-line configuration”) eliminates the need to handle and/or store an eluent sample between an affinity column and an analytical column.

In other embodiments of the present invention, affinity column 11 and analytical column 12 are not in fluid communication with one another. In this embodiment, an eluent sample leaving an affinity column may be collected and/or stored for future use (i.e., for future introduction into analytical column 12). Such an arrangement is also referred to herein as an “off-line configuration.”

As shown above, exemplary apparatus 10 of the present invention may comprise a number of components. A description of individual components and methods of using individual components alone or in combination is provided below.

### I. Apparatus Components

The apparatus of the present invention may comprise, but are not limited to, one or more of the following components.

A. Affinity Column

The present invention is directed to affinity columns, such as exemplary affinity column 11 shown in FIG. 1, comprising one or more of the following components. As used herein, the term “affinity column” includes columns having one or more of the following components, including affinity columns such as immunoaffinity columns.

1. Column Structure

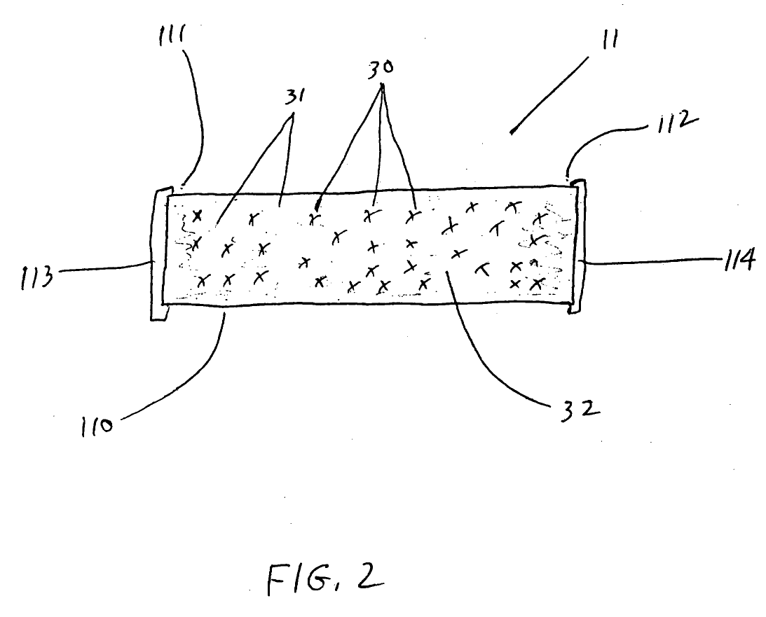

The affinity columns of the present invention comprise a column structure having desired dimensions, column volume, and structural integrity. Typically, the column structure comprises a tubular structure having removable end caps on both ends of the tubular structure. End caps form a leak-proof seal with the tubular structure in order to prevent material from undesirably escaping the tubular structure. An exemplary affinity column 11 of the present invention is shown in FIG. 2.

As shown in FIG. 2, exemplary affinity column 11 comprises tubular structure 110 having first end 111 and second end 112. End caps 113 and 114 form leak-proof seals at first and second ends 111 and 112 respectively. End caps 113 and 114 are particularly useful during storage of exemplary affinity column 11 so as to prevent (i) leakage of materials within exemplary affinity column 11, and/or (ii) drying of materials within exemplary affinity column 11. Exemplary affinity column 11 further comprises rigid support material 30 and first buffer 31 (described below) positioned within a column cavity 32 of exemplary affinity column 11.

Tubular structure 110 may be made from a variety of materials and have a wall construction so as to withstand relatively high pressure within tubular structure 110. Desirably, tubular structure 110 has a structural integrity that withstands a constant pressure of up to about 6000 psi (400 bar), more desirably, from about 3000 psi (200 bar) to about 4500 psi (300 bar). Suitable materials for forming tubular structure 110 include, but not limited to, polymers such as polyetheretherketone (PEEK) and polypropylene; metals such as stainless steel; and inorganic materials such as glass. In one desired embodiment of the present invention, tubular structure 110 comprises polyetheretherketone (PEEK).

Tubular structure 110 may have dimensions that vary depending on a number of factors including, but not limited to, particle size and geometry, flow rate, injection volume, number of required plates, etc. Typically, tubular structure 110 has a circular cross-sectional area, an outer diameter ranging from about 2 mm to about 20 mm, an inner diameter ranging from about 1 mm to about 10 mm, and an overall length ranging from about 2 mm to about 250 mm. In one desired embodiment of the present invention, tubular structure 110 has a circular cross-sectional area, an outer diameter of about 11 mm, an inner diameter of about 4.6 mm, and an overall length of about 50 mm.

End caps 113 and 114 for use with tubular structure 110 are typically formed from PEEK, and have dimensions so as to form a leak-proof seal with ends of tubular structure 110.

It should be noted that although tubular structures having a circular cross-sectional area are desired, tubular structures having other cross-sectional area are also within the scope of the present invention. Suitable cross-sectional configurations for a variety of tubular structures include, but are not limited to, square, rectangular, triangular, oblong, pentagonal and hexagonal cross-sectional configurations.

2. Rigid Support Material

The present invention is further directed to rigid support materials suitable for use in affinity columns, such as exemplary rigid support material 30 shown in FIG. 2. The rigid support materials of the present invention comprise one or more of the following components.

a. Inorganic Substrate

Inorganic substrates suitable for use in the present invention include products commercially available as chromatographic media. The inorganic substrates may be prepared using methods known in the art. The inorganic substrate provides support for one or more additional components applied to a surface of the inorganic substrate. In general, the inorganic substrate is an inorganic oxide, more suitably an inorganic metal oxide, silicate or aluminosilicate or controlled pore glass. An inorganic metal oxide is more desirable. Inorganic oxides suitable for use in the present invention typically have free hydroxyl groups capable of bonding to or reacting with other chemical functionalities. Desirably, the inorganic oxide has about 1 to about 10 hydroxyl groups per square nanometer of solid inorganic oxide.

Suitable inorganic metal oxides include, but are not limited to, silica such as chromatographic grade silica or silica gel, alumina, silica-alumina, zirconia, zirconate, controlled pore glass or titania. In one desired embodiment of the present invention, the inorganic metal oxide is silica, more desirably, chromatographic grade silica or silica gel. Magnetically responsive inorganic metal oxides, such as siliceous oxide-coated magnetic particles disclosed in WO 98/31461 (the disclosure of which is incorporated herein in its entirety by reference) may also be used in the present invention. Mixed inorganic metal oxides, e.g. co-gels of silica and alumina, or co-precipitates may also be used.

The solid inorganic metal oxides may be in a physical form of particulates, fibers plates, or a combination thereof. Desirably, the solid inorganic metal oxides are in a physical form of particulates or particles having a substantially spherical shape. Regardless of the physical form, the solid inorganic metal oxides typically have a longest dimension (i.e., length, width or diameter) of up to about 100 micrometers (μm). When the solid inorganic metal oxide comprises a plurality of particles having a substantially spherical shape, the plurality of particles desirably have an average particle diameter ranging from about 1 μm to about 100 μm. In one desired embodiment of the present invention, the solid inorganic metal oxide comprises a plurality of silica or silica gel particles having a substantially spherical shape, wherein the plurality of silica or silica gel particles have an average particle diameter ranging from about 15 μm to about 20 μm.

A variety of commercially available solid inorganic metal oxides may be used in the present invention. Suitable solid inorganic metal oxides include, but are not limited to, silica particles commercially available from Grace Vydac (Columbia, Md.) under the trade designation DAVISIL®, such as DAVISIL® XWP (extra wide pore) silica media, which are irregular shaped with an average pore size of about 500 Å to about 3000 Å, desirably from about 500 Å to about 1500 Å, or VYDAC® silica having a spheroidal shape and an average pore size of about 300 Å. In one desired embodiment of the present invention, VYDAC® silica having a spheroidal shape and an initial average pore size of about 300 Å is used after being modified to increase the average pore size to about 800 Å.

b. Modified Inorganic Substrate Surface

The surfaces of the above-described inorganic substrates are treated or modified in order to reduce non-specific, non-selective binding and/or adsorption of non-analyte materials (i.e., non-specific binding of materials other than the target analyte) and ligand-specific analyte materials (i.e., non-specific binding of the target analyte to reactive sites other than reactive sites provided by the one or more ligands) onto the inorganic substrate. The resulting modified substrate surface has (i) less affinity for non-analyte materials (i.e., materials other than the target analyte) due to the presence of relatively inert R groups on the inorganic surface, and (ii) a controlled amount of reactive sites for selectively bonding to one or more ligands (described below) to the inorganic substrate surface directly or through a linker. The amount of reactive sites for selectively bonding to one or more ligands leads to selective, controlled binding of one or more analytes of interest to the one or more ligands attached to the inorganic substrate surface.

The modified substrate surface comprises relatively inert R groups attached to at least a portion of the surface of the inorganic surface. As used herein, the term “relatively inert R groups” is used to describe R groups attached to the surface of the inorganic substrate, wherein the R groups have a reactivity of less than the original (i.e., unmodified) inorganic substrate surface. For example, when the inorganic substrate comprises silica particles, the relatively inert R groups attached to at least a portion of the surface of the inorganic surface have a reactivity of less than the hydroxyl groups present on the original or unmodified silica surface.

Relatively inert R groups may be attached to at least a portion of the surface of the inorganic surface using a variety of techniques including, but are not limited to, techniques described herein, as well as techniques described in U.S. patent application Ser. No. 09/929,621, entitled “SOLID COMPOSITIONS FOR SELECTIVE ADSORPTION FROM COMPLEX MIXTURES” filed on Aug. 14, 2001, the subject matter of which is hereby incorporated in its entirety by reference.

In one exemplary embodiment of the present invention, relatively inert R groups are attached to at least a portion of the surface of the inorganic surface, wherein the relatively inert R groups comprise R₁₀ surface moieties. Suitable R₁₀ surface moieties include, but are not limited to, CH₂OH, CH(OH)₂, —CH(OH)CH₃, CH₂CH₂OH, —C(OH)₂CH₃, CH₂CH(OH)₂ and CH(OH)CH₂(OH). In one exemplary embodiment of the present invention, R₁₀ surface moieties comprise CH₂OH, CH(OH)CH₃, CH₂CH₂OH or CH(OH)CH₂(OH). In one desired embodiment of the present invention, R₁₀ surface moieties comprise CH₂OH, CH(OH)CH₃ or CH₂CH₂OH, more desirably, R₁₀ surface moieties comprise CH₂OH.

The moiety R₁₀ is located on at least one surface of the inorganic substance. By “located” it is meant R₁₀ can be attached directly to a functionality on the surface of the starting inorganic substance. R₁₀ can be located on surface area present on the periphery of the inorganic substance, or located on surface area presented in pores, which penetrate into the interior of the inorganic substance and have (pore) openings on the substance's periphery.

R₁₀ can also be “located” on the surface of the inorganic substance by being attached to the inorganic substance surface via bivalent moiety or atom (—X—) to form a group having the formula —X—R₁₀. The bivalent moiety or atom linking R₁₀ in this embodiment is not present in the composition of the starting inorganic substance prior to reaction of the substance with the reactant. The moiety or atom can be from a reactant employed to create R₁₀, e.g., a residual metal atom (e.g. silicon atom), originating from a silane reactant. The residual moiety or atom is attached directly to the inorganic substrate, and desirably through hydroxyl groups on the surface of the inorganic substrate. The —X— groups in such reactants vary from reactant to reactant, but can be metal atoms or other chemical moieties. For example, X can be derived from metal atoms such as silicon, aluminum, zirconium or the like. The inorganic substrate selected may also determine the selection of —X— and its associated reactant. Generally, any reactant containing —X— will be that which can react with reactive functionality on the inorganic substrate. In the case of inorganic oxides, suitable reactants typically are those capable of reacting with hydroxyl groups.

The chemistry of reacting compounds, e.g., those capable of creating R₁₀ on an inorganic substrate, is known in the art, e.g., Smith, Organic Synthesis, John Wiley & Sons, 1994; March, Advanced Organic Chemistry, John Wiley & Sons, Fourth Edition, 1992; Larock, Comprehensive Organic Transformations, John Wiley & Sons, Second Edition, 1999; Greene et al, Protective Groups in Organic Synthesis, John Wiley & Sons, Third Edition, 1999; Brook, Silicon in Organic, Organometallic, and Polymer Chemistry, John Wiley & Sons, 2000; Hermanson et al, Immobilized Affinity Ligand Techniques, 1992; Weetall, “Covalent Coupling Methods for Inorganic Support Materials”, in Methods in Enzymology, vol. XUV, edited by K. Mosbach, pp. 134-148, 1976; Abbott, U.S. Pat. No. 4,298,500; and Arkles, U.S. Pat. No. 5,371,262; the disclosures of each of these documents are herein incorporated in their entirety by reference. For example, a rigid support comprising R₁₀ groups located on the inorganic substance's surface can be prepared from a reactant or coating agent such as alkoxysilane, dialkoxysilane or trialkoxysilane bearing a precursor group of R₁₀. For instance, acetoxymethyl can be the precursor group of hydroxymethyl. The coating agent is then allowed to react with the surface of the inorganic substance, followed by hydrolysis of the precursor to produce an inorganic substance having R₁₀ groups attached.

The modified substrate surface further comprises a controlled amount of reactive sites for selectively bonding to one or more ligands (described below). The reactive sites may be directly on a surface of the inorganic substrate or may be formed via linkers attached to the surface of the inorganic substrate. Ligands may be attached directly to a surface of the inorganic substrate using methods known in the art (e.g. Hermanson et al, Immobilized Affinity Ligand Techniques, Academic Press, 1992 and the other references cited earlier with respect to attaching R₁₀ moieties). For example, the ligand can be attached via a reaction with surface functional groups (i.e., reactive sites), e.g., hydroxyl, on the starting inorganic oxide.

Alternatively, the ligand can be attached to the inorganic substance via a linker attached to the surface of the inorganic substrate (i.e., an alternative reactive site). The linker can be a bivalent chemical group, which is optionally substituted. The optionally substituted bivalent chemical group can comprise n —R— groups, with n being the number of —R— groups, n being an integer of at least 1, preferably not larger than 30, and more preferably not higher than 15. More typically, the bivalent chemical group is about 1 to about 30 atoms, preferably about 1 to about 20 atoms, more preferably about 5 to about 15 atoms, in length measured from the ligand to the inorganic substance. The chemical group —R— can be selected from the group consisting of —C(R₁)H—, —C(R₂)═C(R₃)— and —C≡C—, where R₁, R₂ and R₃ independently being H, alkyl, substituted alkyl, cycloalkyl, substituted cycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, aralkyl or substituted aralkyl, said —R— group optionally replaced with —O—, —S—, carbonyl, thiocarbonyl, —OC(O)—, —C(O)O—, —SC(O)—, —C(O)S—, —OC(S)—, —C(S)O—, —C(S)S—, —SC(S)—, —N(R₄)—, —N(R₄)C(O)—, —C(O)N(R₄)—, —C(R₅)═N—, —N═C(R₅)—, —C(R₅)═NO—, —ON═C(R₅)—, —P—, —P(OH)O—, arylene, substituted arylene, cycloalkylene, substituted cycloalkylene, cycloalkenylene, substituted cycloalkenylene, bivalent heterocyclyl or bivalent substituted heterocyclyl, where R₄ and R₅ are each independently H, alkyl, substituted alkyl, cycloalkyl; substituted cycloalkyl, alkenyl, substituted alkenyl, cycloalkenyl, substituted cycloalkenyl, alkynyl, substituted alkynyl, cycloalkynyl, substituted cycloalkynyl, aryl, substituted aryl, aralkyl or substituted aralkyl. Illustrative of the chemical group is “hydrocarbyl” comprising n —R— groups and wherein n is described above, at least one —R— group is —CH₂ and (n-1)-R— groups are optionally replaced with the R groups mentioned above, e.g., —O—, —S—, etc.

The chemistry of reacting linkers to inorganic substrates is well described in the literature (see Hermanson et al, Immobilized Affinity Ligand Techniques, 1992 and Weetall, Methods in Enzymology, vol. XIV, pp. 134-148, 1976). The particular chemistry for reacting a linker and an inorganic substrate depends on the inorganic substrate and linker employed. Likewise, the chemistry of reacting the linker to a ligand depends on the linker and the ligand employed. Specific examples of suitable linker/ligand coupling chemistry are described in U.S. patent application Ser. No. 09/929,621, entitled “SOLID COMPOSITIONS FOR SELECTIVE ADSORPTION FROM COMPLEX MIXTURES” filed on Aug. 14, 2001, the subject matter of which is hereby incorporated in its entirety by reference. As disclosed in U.S. patent application Ser. No. 09/929,621, for example, a ligand may be coupled to a linker via an amino, sulfhydryl, carbonyl or hydroxy group or an active hydrogen atom on the ligand and/or linker.

In one exemplary embodiment of the present invention, one or more ligands are coupled to an inorganic substrate via a linker having at least one aldehyde functional group thereon. The aldehyde functional group may be used to bond to a ligand, a first linker attached to the inorganic substrate, or both. In one desired embodiment of the present invention, one or more ligands are coupled to an inorganic substrate via a first and second linker, wherein the first linker bonds to the inorganic substrate, and the second linker bonds to the first linker. In one desired embodiment of the present invention, the first linker comprises an amino-functional siloxane, such as aminopropyltrimethoxysilane, and the second linker comprises a dialdehyde, such as glutaraldehyde. In this embodiment, the free aldehyde moiety is used to bind a ligand to the inorganic substrate. This exemplary embodiment of the present invention is described below in Example 1.

In making rigid supports of the present invention having a modified substrate surface, wherein the rigid support comprises linker groups, the order of creating linker groups in conjunction with adding R₁₀ groups to the inorganic substance can vary. The R₁₀ groups can be created on the inorganic surface after attaching a linker, or the R₁₀ groups can be created prior to reacting the inorganic substrate with a linker. Alternatively, precursors to either R₁₀ or the linker or both can be created and/or attached, with the precursors later reacted to create the final R₁₀ and/or linker.

The concentration of linker groups on the modified inorganic surface can vary. In certain embodiments of the present invention, the ligand comprises large protein molecules, which can “shadow” large regions of the rigid support's surface area. As a result, the concentration of the linker sites on the rigid support's surface does not need to be relatively high. The concentration can be optimized based on the size of the contemplated ligand/analyte complex. Factors that determine concentrations of R₁₀ and ligand include, but are not limited to, the identity of R₁₀ groups and ligands, the concentration of reactive sites on the inorganic substance, the concentration of linker groups, and the identity of the analyte.

In general, the concentration of R₁₀ can be in the range of about 1 to about 10 groups per square nanometer (nm²) of rigid support surface area, based on surface area measured by BET. In certain embodiments, the ligand concentration depends primarily on the analyte sought to be recovered when using the composition. As indicated above, the concentration of ligand can also depend on the concentration of any optional linker used. In general, however, the ligand can be in a concentration in the range of 0.04 to about 4 groups per square nanometer. In addition, a given ligand is not always attached to a linker on a one to one stoichiometry. In certain embodiments, e.g., when the ligand is prepared from a large protein molecule, the ligand can be attached by several linker groups. In other embodiments employing smaller ligands, less than stoichiometric amounts of ligands are used and any unreacted linker groups are “capped” to avoid interference when the invention is used for a separation.

The amount of R₁₀ and ligand or optional linker can also be stated in terms of how many functional groups on the starting inorganic substance are reacted or “covered” by (i) the R₁₀ group and (ii) the ligand and/or optional linkers. For example, about 50% to about 99% of reactive sites, such as surface hydroxy groups, on the inorganic substrate can be covered with R₁₀ surface moieties and about 50% to about 1% of the reactive sites can be covered with the ligand and/or optional linker.

In certain embodiments of the present invention, about 70% to about 95% of the reactive sites on the surface of the inorganic substrate is covered with R₁₀ surface moieties and about 30% to about 5% of the reactive sites is covered with the ligand and/or optional linker.

c. Ligands

The rigid support materials of the present invention further comprise one or more ligands bonded to the above-described inorganic substrate. The one or more ligands may be attached directly to reactive sites on the inorganic substrate or optionally via a linker attached to the inorganic substrate as described above. The ligand may be any molecule or molecule fragment capable of binding to another moiety or molecule-based analyte, e.g., binding through hydrophobic interaction, covalent bonding or Columbic interaction. Such ligands are well known to those skilled in the separations industry. Ligands typically used in the bioseparations industry include, but are not limited to, biotin, avidin, streptavidin, natural or unnatural protein, peptide, antigen and nucleic acid. In the present invention, the ligand is preferably a receptor or antibody.

Suitable ligands for use in the present invention include any ligand that selectively bonds to a given analyte. Non-limiting examples of suitable ligands for use in the present invention include, but are not limited to, monoclonal anti-aflatoxin B1 antibodies, monoclonal anti-aflatoxin G1 antibodies, monoclonal anti-aflatoxin Q1 antibodies, monoclonal anti-aflatoxin B2 antibodies, monoclonal anti-aflatoxin G2 antibodies, monoclonal anti-Bisphenol A antibodies, monoclonal anti-2,4-dichlorophenoxy acetic acid antibodies, monoclonal anti-2,4,5-trichlorophenoxy acetic acid antibodies, monoclonal anti-4-chloro-2-methyl acetic acid antibodies, monoclonal anti-4-(2,4-dichlorophenoxy)butyric acid antibodies, monoclonal anti-estrone antibodies, monoclonal anti-17-β-estradiol antibodies, monoclonal anti-17-α-ethynylestradiol antibodies, monoclonal anti-lactoferrin antibodies, monoclonal anti-testosterone antibodies, monoclonal anti-nortestosterone antibodies, monoclonal anti-phenylurea herbicide antibodies (e.g., monoclonal antibodies of metobromuron, cinosulfuron, triasulfuron and/or prosulfuron), monoclonal anti-vinclozolin antibodies, monoclonal anti-folic acid antibodies, monoclonal anti-vitamin B₁₂ (cyanocobalamine) antibodies, monoclonal anti-organophosphor pesticide antibodies (e.g., monoclonal antibodies of fenitrothion, chlorpyrifos and/or pirimifos), anti-catechol amine antibodies (e.g., monoclonal antibodies of adrenaline, noradrenaline and/or dopamine), recombinant human estrogen receptor (hER), and combinations thereof.

As shown in Table 1 below, a variety of ligands may be used to capture a given analyte.

TABLE 1
Exemplary Ligands and Analytes To Be Detected

| | Analyte To Be Detected and |
|---|---|
| Ligand Used To Capture Analyte | Analyzed |
| monoclonal anti-aflatoxin B1 | aflatoxin B1 |
| monoclonal anti-aflatoxin g1 | aflatoxin g1 |
| monoclonal anti-aflatoxin Q1 | aflatoxin Q1 |
| monoclonal anti-aflatoxin B2 | aflatoxin B2 |
| monoclonal anti-aflatoxin G2 | aflatoxin G2 |
| anti-Bisphenol A | Bisphenol A |
| monoclonal anti-2,4-D antibody | chlorophenoxy acetic acids |
| monoclonal anti-estrone | estrone |
| monoclonal anti-17-β-estradiol | 17-β-estradiol |
| monoclonal anti-17-α-ethynylestradiol | 17-α-ethynylestradiol |
| anti-human lactoferrin | lactoferrin |
| monoclonal anti-testosterone | testosterone |
| monoclonal anti-nortestosterone | nortestosterone |
| monoclonal anti-phenylurea antibody | phenylurea herbicides such as |
| | metobromuron. cinosulfuron, |
| | triasulfuron and Prosulfuron |
| monoclonal anti-vinclozolin | vinclozolin |
| monoclonal anti-folic acid | folic acid |
| monoclonal anti-vitamin B₁₂ | vitamin B₁₂ (cyanocobalamine) |
| polyclonal anti-fenitrothion, anti- | fenitrothion, chlorpyrifos and |
| chlorpyrifos and anti-pirimifos | pirimifos |
| recombinant human estrogen receptor | any compound exhibiting |
| (hER) | estrogenic activity |

The above exemplary ligands are commercially available from a number of sources. Suitable commercially available ligands for use in the present invention include, but are not limited to, ligands shown in Table 2 below.

TABLE 2
Exemplary Commercially Available Ligands

| Ligand Used To | | Product |
|---|---|---|
| Capture Analyte | Source | Name/Designation |
| monoclonal anti- | Sigma-Aldrich | A9555 |
| aflatoxin B1 | (St. Louis, MO) | |
| monoclonal anti- | Sigma-Aldrich | A9555 |
| aflatoxin g1 | (St. Louis, MO) | |
| monoclonal anti- | Sigma-Aldrich | A9555 |
| aflatoxin Q1 | (St. Louis, MO) | |
| monoclonal anti- | Sigma-Aldrich | A9555 |
| aflatoxin B2 | (St. Louis, MO) | |
| monoclonal anti- | Sigma-Aldrich | A9555 |
| aflatoxin G2 | (St. Louis, MO) | |
| monoclonal anti- | COSMO BIO CO., LTD. | FKA-606-E |
| Bisphenol A | (Tokyo, JAPAN) | |
| monoclonal anti- | EltiSupport | Anti-2,4-D |
| 2,4-D antibody | (Malden, the | |
| | Netherlands) | |
| monoclonal anti- | COSMO BIO CO., LTD. | FKA-210, FKA-210-E, |
| estrone | (Tokyo, JAPAN) | FKA-212, FKA-212-E, |
| | | FKA-214, FKA-214-E |
| monoclonal anti- | COSMO BIO CO., LTD. | FKA-204, FKA-236, |
| 17-β-estradiol | (Tokyo, JAPAN) | FKA-236-E |
| monoclonal anti- | COSMO BIO CO., LTD. | FKA-220, FKA-220-E, |
| 17-α- | (Tokyo, JAPAN) | FKA-608-E |
| ethynylestradiol | | |
| anti-human | Sigma-Aldrich | L3262 |
| lactoferrin | (St. Louis, MO) | |
| monoclonal anti- | COSMO BIO CO., LTD. | FKA-118, FKA-118-E, |
| testosterone | (Tokyo, JAPAN) | FKA-128, FKA-128-E |
| monoclonal anti- | COSMO BIO CO., LTD. | FKA-120 and FKA- |
| nortestosterone | (Tokyo, JAPAN) | 120-E |
| monoclonal anti- | EltiSupport | 2/C8/C8 IgG |
| phenylurea | (Malden, the | |
| antibody | Netherlands) | |
| monoclonal anti- | Rikilt | 30-1D2G4G7 |
| vinclozolin | (Wageningen, the | |
| | Netherlands) | |
| monoclonal anti- | R-Biopharm | Anti-folic acid |
| folic acid | (Glasgow, UK) | |
| monoclonal anti- | Sigma-Aldrich | V9505 |
| vitamin B₁₂ | (St. Louts, MO) | |
| recombinant human | Axxora, LLC | ALX-201-033 |
| estrogen receptor | (San Diego, CA) | |
| (hER) | | |

In one desired embodiment of the present invention, the ligand comprises an antibody capable of selectively bonding a mycotoxin from a complex mixture. In this embodiment, the ligand desirably comprises a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, or a combination thereof. Further, in this embodiment, complex mixtures may include, but are not limited to, nuts and nut products, cereals, dried fruit, herbs, spices, coffee, cocoa, coconut, animal feed, vegetable oil, beer, water, biological fluids, etc.

In a further desired embodiment of the present invention, the ligand comprises an antibody capable of selectively bonding folic acid, vitamin B₁₂ (cyanocobalamine), or both from a complex mixture. In this embodiment, the ligand desirably comprises a monoclonal anti-folic acid antibody, a monoclonal anti-vitamin B₁₂ (cyanocobalamine) antibody, or a combination thereof. Further, in this embodiment, complex mixtures may include, but are not limited to, food samples, (e.g., infant formula, pet food, sport drink, and vitamin tablets), biological samples (e.g., animal tissue, biological samples, etc.).

In yet a further desired embodiment of the present invention, the ligand comprises the native estrogen receptor, the recombinant estrogen receptor or any derivative thereof, a recombinant protein and/or any other ligand mimicking the biological active part of the receptor capable of selectively bonding one or more endocrine disrupters from a complex mixture. The term “endocrine disrupters” is used to identify a class of compounds that are suspected of interfering with the endocrine system of human beings and wildlife. “Endocrine disrupters” (also called “xeno-estrogens”) disrupt the hormonal balance and can have deleterious effects in humans, animals, and their offspring. Known exemplary endocrine disrupters include, but are not limited to, Bisphenol A, estrone, 17-α-estradiol, 17-β-estradiol, 17-α-ethynylestradiol, alkylphenols, diethylstilbestrol, methoxychlor, zearalenone, genistein, o,p′-DDT, p,p′-DDT and butylbenzyl phthalate. However, there are believed to be many unknown endocrine disrupters that potentially interfere with the functions of normal endocrine systems of human beings and wildlife. Therefore, this embodiment of the present invention may be useful in identifying one or more known or unknown endocrine disrupters in a complex mixture. In this embodiment, complex mixtures may include, but are not limited to, human and bovine biological fluids (such as serum and urine), tap water, ground water, process waters, environmental samples like ground, sludge, surface waters in general and surface waters containing possible pharmaceutical contamination in particular, industrial chemical formulations, contaminated food due to leakage of chemicals from packaging materials, and packaging materials.

In this embodiment, the ligand desirably comprises an estrogen receptor. The estrogen receptor ligand extracts compounds having estrogenic activity from complex mixtures, while having essentially no affinity for compounds in the mixture that do not have estrogenic activity. As used herein, the term “compound(s) having estrogen activity” refers to compounds that are defined as endocrine disrupter (e.g., an exogenous agent that interferes with the production, release, transport, metabolism, binding, action or elimination of natural hormones in the body responsible for the maintenance of homeostasis and the regulation of developmental processes, Kavlock et al., “Research needs for the risk assessment of health and environmental effects of endocrine disruptors: A report of the U.S. EPA-sponsored workshop.” Environ. Health Perspect. 104 Suppl 4:715-740 (1996)) and described in an Endocrine Disruptor Knowledge Base (EDKB) accessible from an FDA public website: http://edkb.fda.pov. Suitable estrogen receptor ligands for use in the present invention include, the native human estrogen receptor, a recombinant human estrogen receptor (hER) or derivatives thereof, a recombinant protein mimicking the biological active part of the estrogen receptor or derivatives thereof or any ligand, which selectively recognises compounds on their biological activity as endocrine disrupter. Desirably, the estrogen receptor ligand comprises a recombinant human estrogen receptor (hER).

In yet a further desired embodiment of the present invention, the ligand comprises an antibody capable of selectively bonding one or more steroid hormones from a complex mixture. Steroid hormones include, but are not limited to, estradiol, estrone, ethynylestradiol, testosterone and nortestosterone. In this embodiment, complex mixtures may include, but are not limited to, tap water, ground water, process waters, environmental samples like ground, sludge, surface waters in general and surface waters containing possible pharmaceutical contamination in particular, pharmaceutical formulations, human and animal biological fluids (such as serum and urine), and other biological samples (e.g., animal tissue, biological samples, etc.).

The affinity columns of the present invention desirably possess a minimum analyte capture capacity. The desired analyte capture capacity for a given affinity column may vary depending on a number of factors including, but not limited to, the content and type of analyte, the available test sample size, sensitivity and limits of detection of the measuring device, etc. Typically, the affinity columns of the present invention are capable of capturing up to about 500 picomoles (pMol) of a given analyte. Desirably, affinity columns are capable of capturing from about 50 pMol to about 1000 pMol of a given analyte.

3. Buffer Solution

The affinity columns of the present invention may further comprise a buffer solution, such as exemplary first buffer 31 shown in FIG. 2. Suitable first buffer solutions provide a non-reactive protective media for the rigid support material within the affinity column during storage and/or use of the affinity column. Suitable first buffer solutions for use in the present invention include, but are not limited to, phosphate buffered saline (PBS) buffer or a PBS buffer containing about 0.02 wt % sodium azide. Specific first buffer solutions include, but are not limited to, a 0.01 M phosphate+0.15 M NaCl buffer having a pH of about 7.0.

Typically, the first buffer solution has a pH ranging from about 6.0 to about 8.0. The first buffer solution desirably has a pH ranging from about 6.8 to about 7.4, more desirably, a pH ranging from about 7.0 to about 7.4, and even more desirably, a pH of about 7.0.

Desirably, the first buffer solution comprises PBS buffer containing about 0.02 wt % sodium azide during storage of the affinity column containing a rigid support material as described above. The affinity columns are desirably stored at a temperature ranging from about +4° C. to about +8° C. in the PBS buffer. Further, first buffer solution desirably comprises PBS buffer having a pH of about 7.0 during use of the affinity column containing a rigid support material.

B. Analytical Column

The apparatus of the present invention may further comprise one or more analytical columns such as exemplary analytical column 12 shown in FIG. 1. Each analytical column may be used to capture one or more analytes present in an eluent sample. Any commercially available analytical column may be used in the present invention in combination with any of the above-described apparatus components.

Suitable commercially available analytical columns include, but are not limited to, analytical columns available from Grace GmbH & Co. KG (Worms, Germany) under the trade designations GENESIS® and DENALI™ such as GENESIS® C8 e/c having a variety of sizes including 5 μm, 4.6×250 mm; and 5 μm, 4.6×150 mm; GENESIS® C18 having a variety of sizes including 4 μm, 4.6×250 mm; and DENALI™ C18 having a variety of sizes including 5 μm, 4.6×150 mm; analytical columns available from Grom Analytik+HPLC GmbH (Rottenburg-Hailfingen, Germany) under the trade designation GROM-Sil, such as GROM-Sil ODS type columns having a variety of sizes including 5 μm, 4.6×150 mm; and cation exchange columns available from Amersham Biosciences (Uppsala, Sweden) under the trade designation Mono S, such as Mono S hr5/5 having a variety of sizes including 10 μm, 1 ml.

In one desired embodiment of the present invention, an analytical column is connected to an affinity column such that the affinity column is in fluid communication with the analytical column. In this embodiment, it is desirable for the tubular structure of the analytical column to be made from materials and have a wall construction sufficient to withstand relatively high pressure within the tubular structure (i.e., up to about 6000 psi (400 bar), more desirably, from about 3000 psi (200 bar) to about 4500 psi (300 bar)). Suitable tubular structure materials include the above-described materials for forming a tubular structure of an affinity column of the present invention.

Typically, the analytical column forms part of a liquid chromatography device, such as a high pressure liquid chromatography (HPLC) device. Suitable liquid chromatography equipment for use in the present invention includes, but is not limited to, liquid chromatography equipment commercially available from companies such as Shimadzu (Columbia, Md.), Agilent Technologies (Wilmington, Del.), Applied Biosystems (Fostercity, Calif.), Dionex Corporation (Sunnyvale, Calif.), Varian Inc. (Palo Alto, Calif.), and Waters Inc. (Milford, Mass.).

C. Detector

The apparatus of the present invention may further comprise one or more detectors such as exemplary detector 13 shown in FIG. 1. Detectors may be used to detect and quantify analytes present in a mobile phase sample. Any commercially available detector may be used in the present invention in combination with any of the above-described apparatus components.

Suitable commercially available detectors include, but are not limited to, UV-VIS detectors available from Shimadzu, Inc. (Columbia, Md.), such as the Series SPD10 UV/Vis Detector, or other types of detectors such as, but not limited to, fluorescence detectors, refractive index detectors, mass-selective detectors and electrochemical detectors, which are commercially available from companies such as, but not limited to, Agilent Technologies (Wilmington, Del.), Applied Biosystems (Fostercity, Calif.), Dionex Corporation (Sunnyvale, Calif.), Varian Inc. (Palo Alto, Calif.), and Waters Inc. (Milford, Mass.). Desirably, the detector comprises a UV-VIS detector operating at a wavelength ranging from about 230 nanometers (nm) to about 400 nm. For example, the following exemplary wavelengths are useful in the present invention: UV-VIS at 230 nm; UV-VIS at 240 nm (vinclozolin); and UV-VIS at 361 nm (vitamin B₁₂).

D. Coupling

The apparatus of the present invention may further comprise a coupling between the affinity column and one or more analytical columns. Any coupling material may be used in the present invention that is conventionally used in chromatography processes. Typically, the coupling comprises low dead volume connections from plastic, metal or glass tubing. In embodiments of the present invention wherein the affinity column is in fluid communication with the analytical column, the coupling is made from materials and has a wall construction sufficient to withstand relatively high pressure within the coupling (i.e., up to about 6000 psi (400 bar), more desirably, from about 3000 psi (200 bar) to about 4500 psi (300 bar)).

E. Pumps

The apparatus of the present invention may further comprise one or more pumps such as exemplary first pump 14 and second pump 15 shown in FIG. 1. Each pump provides fluid flow through the above-described apparatus components. Any commercially available pump may be used in the present invention in combination with any of the above-described apparatus components.

Suitable commercially available pumps include, but are not limited to, pumps available from Shimadzu (Columbia, Md.), Agilent Technologies (Wilmington, Del.), Applied Biosystems (Fostercity, Calif.), Dionex Corporation (Sunnyvale, Calif.), Varian Inc. (Palo Alto, Calif.), and Waters Inc. (Milford, Mass.). Desirably, the pumps comprise programmable low or high pressure gradient pumps having at least three channels commercially available from Agilent Technologies (Wilmington, Del.) under the trade designation 1100 Series, such as the quaternary model 1100 pump.

In one desired embodiment of the present invention, a first pump is used to provide fluid flow of the first buffer and a test sample through the affinity column, while a second pump is used to provide fluid flow of an elution buffer solution and an eluent sample through the analytical column.

F. Valves

The apparatus of the present invention may further comprise one or more valves such as exemplary first valve 16 and second valve 17 shown in FIG. 1. Each valve controls fluid flow through the above-described apparatus components. Any commercially available valve may be used in the present invention in combination with any of the above-described apparatus components.

Suitable commercially available valves include, but are not limited to, valves available from VICI Valco Instruments Co., Inc. (Houston, Tex.) or VWR International Ltd. (Dorset, UK). Desirably, the values comprise programmable two-position six-way valves (herein referred to as programmable six-way valves) commercially available from VWR International Ltd. (Dorset, UK) under the trade designation RHEODYNE, such as model 7725 sample injector.

In one desired embodiment of the present invention, a first programmable six-way valve is used to control fluid flow of the first buffer and/or a test sample through the affinity column, while a second programmable six-way valve is used to control fluid flow of an elution buffer solution and an eluent sample through the analytical column.

### II. Methods of Making Apparatus Components

The present invention is further directed to methods of making the above-described apparatus components. Rigid support materials, for example, may be made as described above and in the examples below. In general, the method of making a rigid support material of the present invention comprises the following steps:

(1) modifying an outer surface of an inorganic substrate in order to reduce non-specific, non-selective binding and/or adsorption of non-analyte materials onto the inorganic substrate; and

(2) selectively bonding one or more ligands to the inorganic substrate surface.

As described above, the step of modifying the inorganic substrate surface comprises (i) attaching relatively inert R groups to at least a portion of the surface of the inorganic substrate, and optionally (ii) attaching one or more linkers to at least a portion of the surface of the inorganic substrate. The step of attaching relatively inert R groups to at least a portion of the surface of the inorganic substrate may take place prior to or after the optional step of attaching one or more linkers to at least a portion of the surface of the inorganic substrate. The step of selectively bonding one or more ligands to the inorganic substrate surface may comprise (i) bonding a controlled amount of one or more ligands directly to reactive sites on the inorganic substrate surface, or (ii) bonding a controlled amount of one or more ligands to one or more linkers extending from the inorganic substrate surface. When a controlled amount of one or more ligands is bonded directly to reactive sites on the inorganic substrate surface, the step of selectively bonding one or more ligands to the inorganic substrate surface may occur prior to or after the step of attaching relatively inert R groups to at least a portion of the surface of the inorganic substrate.

In one desired embodiment of the present invention, the method of making a rigid support material comprises the following steps:

(1) attaching R groups to at least a first portion of the surface of the inorganic substrate, wherein the R groups have a reactivity less than any functional groups on the surface of the inorganic substrate prior to the attaching step;

(2) attaching one or more linkers to at least a second portion of the surface of the inorganic substrate; and

(3) selectively bonding one or more ligands to the one or more linkers. Steps (1) and (2) may be conducted in any order. Desirably, the one or more linkers comprise an amino-substituted siloxane in combination with a dialdehyde. More desirably, the one or more linkers comprise aminopropyltrimethoxysilane in combination with glutaraldehyde.

Affinity columns of the present invention may be prepared using the following steps:

(1) sealing a first end of a tubular structure;

(2) at least partially filling a column cavity of the tubular structure with a rigid support material, such as any of the above-described rigid support materials;

(3) at least partially filling the column cavity of the tubular structure with a first buffer solution to encapsulate the rigid support material; and, optionally

(4) sealing the opposite end (i.e., the second end) of the tubular structure. The affinity column may be stored for future use or may be subsequently connected to an apparatus comprising one or all of the above-described apparatus components.

### III. Methods of Analyzing Samples

The present invention is even further directed to methods of analyzing test samples that potentially contain one or more analytes of interest. In one exemplary embodiment of the present invention, the method comprises the step of (a) introducing the test sample into an affinity column containing a rigid support, wherein the rigid support comprises a plurality of inorganic metal oxide particles, wherein each particle comprises (i) a metal oxide substrate; (ii) a modified substrate surface that reduces non-specific binding of non-analyte materials (i.e., non-specific binding of materials other than the target analyte) and ligand-specific analyte materials (i.e., non-specific binding of the target analyte to reactive sites other than reactive sites provided by one or more ligands) to the inorganic substrate; and (iii) one or more ligands bonded to the inorganic substrate.

The methods of analyzing test samples of the present invention may use a variety of ligands including, but not limited to, a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof.

The method of analyzing a test sample may further comprise the steps of (b) allowing the test sample to come into contact with the rigid support and ligands thereon; (c) rinsing the rigid support to wash away any test sample components that do not bond to the ligands; (d) introducing an eluent solution into the affinity column so that the eluent solution comes into contact with one or more analytes bound to the ligands on the rigid support; (e) allowing the eluent solution to remain in contact with the rigid support for a period of time so as to form an eluent sample potentially containing one or more analytes; and (f) analyzing contents of the analytical column to determine a presence of one or more analytes in the test sample.

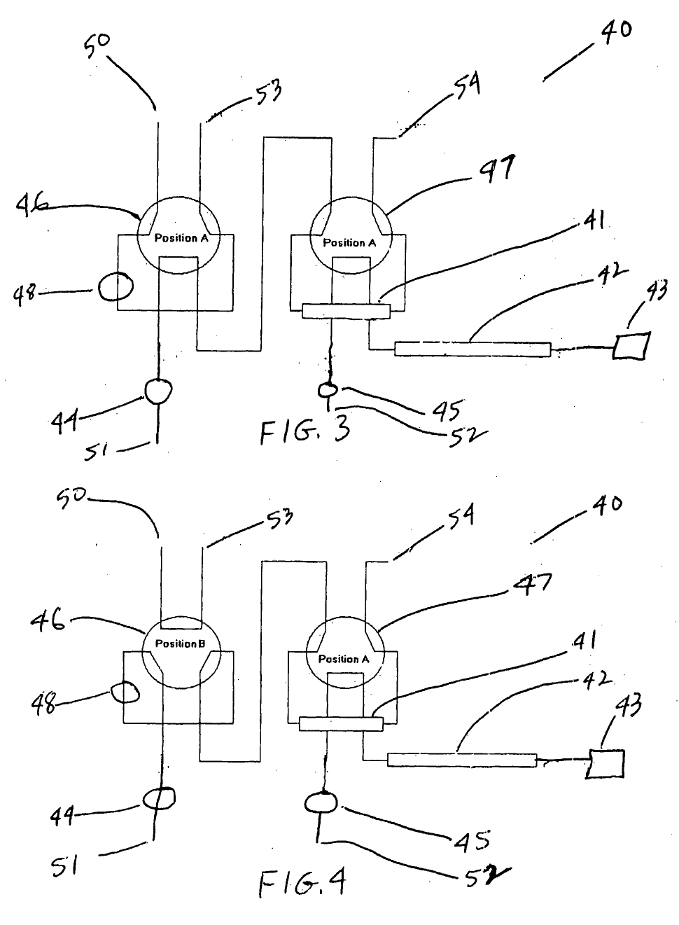

FIGS. 3-6 depict various steps in an exemplary method of analyzing a test sample using one or more of the above-described apparatus components. As shown in FIGS. 3-6, exemplary apparatus 40 comprises affinity column 41, analytical column 42, detector 43, first pump 44, second pump 45, first valve 46, second valve 47, test sample loop 48, test sample inlet 50, first buffer inlet 51, elution buffer inlet 52, first waste outlet 53, and affinity column waste outlet 54. In this embodiment of the present invention, affinity column 41 and analytical column 42 are in fluid communication with one another. Further, test sample loop 48 is utilized to temporarily store a test sample prior to merging the test sample with a first buffer flowing through affinity column 41.

FIG. 3 displays exemplary apparatus 40 during a test sample loading step. A test sample is loaded into test sample inlet 50. As shown in FIG. 3, during this step, programmable six-way first valve 46 is in “Position A” and programmable six-way second valve 47 is in “Position B,” which enables (i) a test sample to flow from test sample inlet 50 to test sample loop 48, and (ii) a first buffer to flow through affinity column 41. Possible test samples may contain any of the above-mentioned analytes in a complex mixture. Suitable first buffer solutions that may be used in exemplary apparatus 40 include any of the above-described first buffer solutions.

In a separate step, the test sample flows through affinity column 41 as shown in FIG. 4. During this step, programmable six-way first valve 46 is in a “Position B” and programmable six-way second valve 47 is in “Position A,” which enables (i) the test sample with first buffer to flow from test sample loop 48 to and through affinity column 41, (ii) fluid flow from test sample inlet 50 to proceed to first waste outlet 53, and (iii) an elution buffer solution to flow from elution buffer inlet 52 through analytical column 42, but not through affinity column 41.

A variety of elution buffer solutions may be used during this step. Suitable elution buffer solutions effectively release analytes bonded to rigid support material as the elution buffer solution travels through affinity column 41. Suitable elution buffer solutions for use in the present invention include, but are not limited to, 0.1 M glycine pH 2.5, 5 M NaCl, 10 mM phosphate, pH 7.2, 3.5 M MgCl₂, 10 mM phosphate, pH 7.2, 2 to 8 M urea, 2 M guanidine HCl, 3 to 5 M thiocyante, 10% dioxane, 50% ethylene glycol, acetonitrile-containing aqueous solutions, and combinations thereof. Specific elution buffer solutions include, but are not limited to, a 35% v/v acetonitrile/65% v/v water elution buffer solution (e.g., for Bisphenol A, 17-α-estradiol, 17α-ethynylestradiol, testosterone, and nortestosterone); 10% v/v acetonitrile/90% v/v water elution buffer solution (e.g., for a chlorophenoxy acetic acid herbicide); a 0.01 M HCl+0.15 M NaCl buffer solution (e.g., for lactoferrin and vitamin B₁₂); and a 10% v/v methanol in 0.01 M HCl+0.15 M NaCl elution buffer solution (e.g., for vinclozolin).

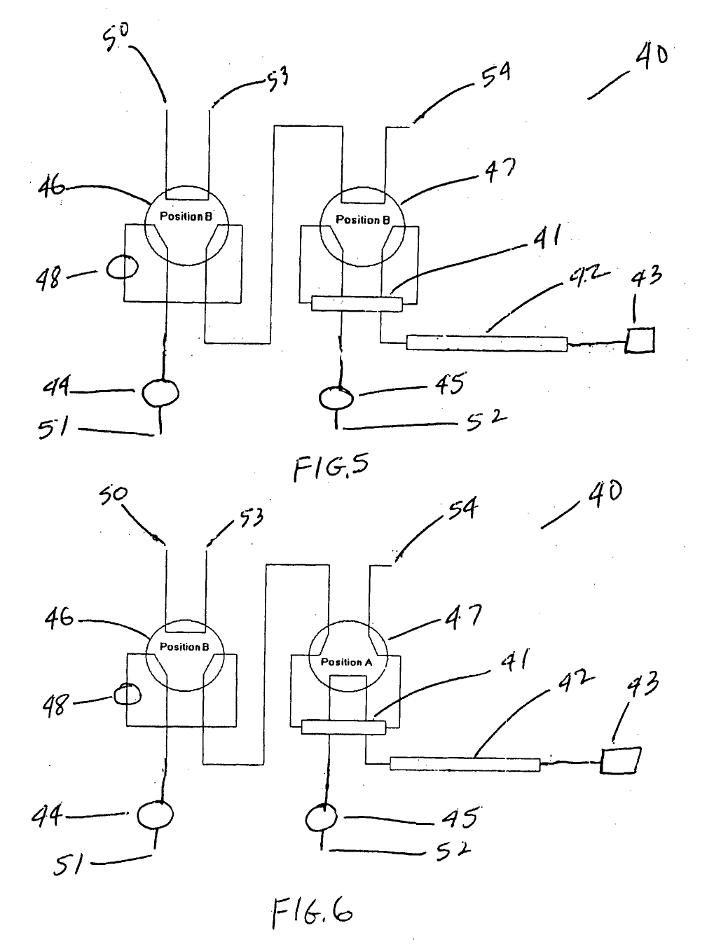

In a further separate step shown in FIG. 5, the elution buffer solution flows through affinity column 41 and analytical column 42. During this step, programmable six-way first valve 46 is in “Position B” and programmable six-way second valve 47 is in “Position B,” which enables (i) fluid flow from first buffer inlet 51 to affinity column waste outlet 54, (ii) fluid flow from test sample inlet 50 to first waste outlet 53, and (iii) the elution buffer solution to flow from elution buffer inlet 52 through affinity column 41 and then directly into analytical column 42.

In another separate step shown in FIG. 6, a mobile phase solution flows through analytical column 42. During this step, programmable six-way first valve 46 is in “Position B” and programmable six-way second valve 47 is in “Position A,” which enables (i) fluid flow from first buffer inlet 51 through affinity column 41 to affinity column waste outlet 54, (ii) fluid flow from test sample inlet 50 to first waste outlet 53, and (iii) the mobile phase solution to flow from elution buffer inlet 52 through analytical column 42 to detector 43.

A variety of mobile phase solutions may be used in the present invention. Suitable mobile phase solutions effectively release analytes bonded to support structures in analytical column 42 as the mobile phase solution travels through analytical column 42. Suitable mobile phase solutions for use in the present invention include, but are not limited to, methanol or acetonitrile-containing aqueous solutions, a HCl solution, a methanol/HCl solution, a phosphate/NaCl solution, a sodium acetate solution, a methanol/sodium acetate solution, an acetonitrile/HCl solution, and a methanol/HCl/NaCl solution, and combinations thereof.

Specific mobile phase solutions suitable for use in the present invention include, but are not limited to, a 45% v/v acetonitrile/55% v/v water mobile phase solution (e.g., for Bisphenol A analyte); 0.01 M HCl mobile phase solution (e.g., for a chlorophenoxy acetic acid herbicide analyte); 60% v/v methanol in 0.01 M HCl mobile phase solution (e.g., for a chlorophenoxy acetic acid herbicide analyte); a 70% v/v acetonitrile/30% v/v water (e.g., for 17-α-estradiol, 17-α-ethynylestradiol, testosterone, and nortestosterone); a 0.10 M phosphate+1.5 M NaCl (pH 7.0) mobile phase solution (e.g., for lactoferrin); a 50 mM sodium acetate (pH 6.0) mobile phase solution (e.g., for phenylurea herbicide); a 55% v/v methanol in 50 mM sodium acetate (pH 6.0) mobile phase solution (e.g., for phenylurea herbicide); a 64% v/v acetonitrile in 0.01 M HCl mobile phase solution (e.g., for vinclozolin); and a 30% v/v methanol/70% v/v 0.01 M HCl+0.15 M NaCl mobile phase solution (e.g., for vitamin B₁₂).

In one desired embodiment of the present invention, the method of analyzing a sample comprises a method of analyzing an eluent sample, wherein the method comprises the steps of transferring the eluent sample from an affinity column to an analytical column, wherein the affinity column is in fluid communication with the analytical column, and analyzing contents of the analytical column to determine a presence of one or more analytes in the eluent sample. For example, the method of analyzing an eluent sample may be used to analyze a sample potentially containing one or more analytes selected from the group consisting of aflatoxin B1, aflatoxin g1, aflatoxin Q1, aflatoxin B2, aflatoxin G2, Bisphenol A, 2,4-dichlorophenoxy acetic acid, 2,4,5-trichlorophenoxy acetic acid, 4-chloro-2-methyl acetic acid, 4-(2,4-dichlorophenoxy)butyric acid, estrone, 17-β-estradiol, 17-α-ethynylestradiol, lactoferrin, testosterone, nortestosterone, metobromuron, cinosulfuron, triasulfuron, Prosulfuron, vinclozolin, folic acid, vitamin B₁₂ (cyanocobalamine), fenitrothion, chlorpyrifos, pirimifos, adrenalin, noradrenalin, dopamine, an endocrine disrupter (e.g., a compound having estrogenic activity), and combinations thereof.

In this embodiment, the method of analyzing an eluent sample wherein the affinity column is in fluid communication with the analytical column, the method may further comprise one or more of the following steps:

(1) introducing a test sample into an affinity column containing a rigid support capable of withstanding a column pressure of up to about 200 bar, wherein the rigid support has one or more ligands bonded thereto, wherein the one or more ligands are capable of selectively bonding to one or more analytes;

(2) allowing the test sample to come into contact with the rigid support and ligands thereon;

(3) rinsing the rigid support to wash away any test sample components other than the one or more analytes;

(4) introducing an eluent solution into the affinity column so that the eluent solution comes into contact with the one or more analytes bound to the ligands on the rigid support; and

(5) allowing the eluent solution to remain in contact with the rigid support for a period of time so as to form the eluent sample. Typically, the eluent solution remains in contact with the rigid support for a period of time ranging from about 5 minutes to about 15 minutes.

In one exemplary method of analyzing an eluent sample, the method comprises a method of analyzing an eluent sample that potentially contains a mycotoxin. In this exemplary method, the method comprises the steps of transferring the eluent sample from an affinity column to an analytical column, wherein the affinity column is in fluid communication with the analytical column, and analyzing contents of the analytical column to determine a presence of least one mycotoxin in the eluent sample. The eluent sample may be analyzed for the presence of aflatoxin B1, aflatoxin g1, aflatoxin Q1, aflatoxin B2, aflatoxin G2, or a combination thereof.

In a further exemplary method of analyzing an eluent sample, the method comprises a method of analyzing an eluent sample that potentially contains folic acid, vitamin B₁₂ (cyanocobalamine), or a combination thereof, wherein the method comprises the steps of transferring the eluent sample from an affinity column to an analytical column, wherein the affinity column is in fluid communication with the analytical column, and analyzing contents of the analytical column to determine a presence of folic acid, vitamin B₁₂ (cyanocobalamine), or both in the eluent sample.

The present invention is further directed to methods of analyzing test samples, wherein the test sample potentially contains at least one compound having estrogen activity. In this embodiment, the method comprises the steps of introducing the test sample into an affinity column containing a rigid support having one or more ligands bonded thereto, wherein the one or more ligands are capable of selectively bonding to one or more compounds having estrogen activity, such as the native human estrogen receptor, a recombinant human estrogen receptor (hER) or derivatives thereof, a recombinant protein mimicking the biological active part of the estrogen receptor or derivatives thereof or any ligand which selectively recognises compounds on their biological activity as endocrine disrupter. In one desired embodiment, the one or more ligands comprise recombinant human estrogen receptor (hER).

The exemplary method of analyzing test samples that potentially contain at least one compound having estrogen activity may further comprise one or more of the following steps:

(1) allowing the test sample to come into contact with the rigid support and ligands thereon;

(2) rinsing the rigid support to wash away any test sample components that do not exhibit estrogen activity;

(3) introducing an eluent solution into the affinity column so that the eluent solution comes into contact with the one or more compounds having estrogen activity bound to the ligands on the rigid support;

(4) allowing the eluent solution to remain in contact with the rigid support for a period of time so as to form a eluent sample containing the compounds having estrogen activity; and

(5) analyzing contents of the analytical column to determine a presence of one or more compounds having estrogen activity in the eluent sample. In one desired variation of this embodiment, the rigid support is capable of withstanding a column pressure of up to about 200 bar, and the affinity column is in fluid communication with the analytical column.

The affinity columns of the present invention are reusable. Therefore, any of the above described exemplary methods may further include one or more of the following steps:

(1) flushing the affinity column with a first buffer solution; and

(2) introducing a second test sample into the affinity column.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

### Preparation of a Rigid Support Comprising Monoclonal Anti-Vitamin B₁₂ Antibody

An exemplary rigid support was prepared as follows. A solution of 500 g toluene and 1.52 g 3-aminopropyltriethoxysilane were added to a 1000 ml round bottom flask. 100 g of Grace Vydac silica having an enlarged average pore size of 800 Å (average particle size of about 15 to about 20μ) that was previously calcined 2 hours at 200° C. was added to the round bottom flask. The round bottom flask was put in a heating mantle and a condenser was attached. The heating mantle was attached to the top of an orbital shaker, which was operated at a speed of 115 rpm. N₂ was passed through the round bottom flask and condenser to remove air during the entire reaction.

The contents of the round bottom flask were heated to boiling (˜110° C.) for 4 hours. The sample was filtered and washed with 2×100 ml toluene, dried at 115° C. and then calcined for 2 hours at 150° C. The resulting sample was labeled Intermediate A.

The concentration of the —C₃H₆NH₂ groups on the silica was calculated to be 0.54 and was based on the surface area (BET) of the silica support (43 m²/g), carbon content (LECO) of the intermediary (0.41%). See ASTM D5373 (for coal) and ASTM 5291.

400 ml 1 M NaCl was mixed with the Intermediate A in a beaker and stirred with a magnetic stirrer. The initial pH was 4.79. 1 M HCl was added dropwise until the pH reached 2.0. The pH was held at 2.0 for 15 minutes. The sample was then filtered and washed with 5×100 ml DI H₂O, dried at 115° C. and then calcined for 2 hours at 200° C. This sample was labeled Intermediate B.

400 g toluene and 48.78 g acetoxymethyltriethoxysilane were mixed with Intermediate B in a round bottom flask. The round bottom flask was placed in a heating mantle with an attached condenser. The heating mantle was attached to the top of an orbital shaker operating a speed of 115 rpm. N₂ was passed through the round bottom flask and condenser to remove air during the entire reaction. The sample was heated to boiling (−10° C.) for 24 hours, filtered, washed with 3×100 ml toluene, dried at 115° C. and then calcined for 2 hours at 150° C. This sample was labeled Intermediate C.

In the next reaction step, 20 g of Intermediate C was added to 80 ml of 0.1 M hydrochloric acid and boiled for 4 hours. The silica was filtered off and washed 4 times with 60 ml deionized water. This sample was labeled Intermediate D.

20 g of Intermediate D and 300 ml of coupling buffer (0.1 M Na₂PO₄+0.15 M NaCl; pH=7.0) were mixed in a 1000 ml beaker and stirred for 5 minutes. The sample was filtered to form a moist cake. Then, 57.53 g of 50 wt. % glutaraldehyde and 2.89 g of NaCNBH₃ were added to the beaker followed by addition of the moist filter cake of Intermediate D. The sample was stirred for 4 hours, filtered, washed with 400 ml coupling buffer and reslurried in 400 ml coupling buffer to obtain a new sample, which was filtered, washed with 200 ml coupling buffer and reslurried in 200 ml coupling buffer 2 more times. The re-washed and reslurried sample was filtered and then washed with 400 ml coupling buffer. This sample was labeled Intermediate E.

1.5 g of coupling buffer and 250 μl of monoclonal anti-vitamin B₁₂ antibody (Product No. V9505 commercially available from Sigma-Aldrich (St. Louis, Mo.)) having a concentration of approximately 10 to 15 mg antibody per milliliter) were added to a 10 ml round bottom flask. 160 mg NaCNBH₃ and 1 gram of Intermediate E were added to the flask and mixed on a shaker for 4 hours. The sample was filtered and washed 4 times with 10 ml of coupling buffer. Then, 1.5 g of coupling buffer, 160 mg of NaCNBH₃ and 50 mg of ethanolamine were added to the 10 ml round bottom flask, and then mixed on a shaker for 4 hours. The sample was filtered and washed 4 times with 10 ml of coupling buffer. The resulting rigid support material was placed in PBS buffer containing 0.02% sodium azide and stored at 4° C.

### EXAMPLE 2

### Preparation of an Affinity Column For Detecting Vitamin B₁₂

An exemplary affinity column was prepared by packing a 4.6×50 mm I.D. affinity column with the rigid support material produced in Example 1. The packed column was then filled with a phosphate buffer solution (pH 7.4) containing 0.02 wt % sodium azide. The resulting exemplary affinity column was stored at a temperature of 4° C.

### EXAMPLE 3

### Analysis of a Vitamin B₁₂-Containing Composition

The exemplary affinity column of Example 2 was coupled to an apparatus similar to exemplary apparatus 40 as shown in FIG. 3. The apparatus comprised a High Performance Liquid Chromatograph (HPLC) (model 1100 series, Agilent Technologies, Wilmington, Del.). Injection was performed with a model RHEODYNE 7725i sample injector (VWR International Ltd., Dorset, UK) equipped with a 200 μl sample loop. By means of a model RHEODYNE 7725 sample injector, it was possible to switch the affinity column from an on-line configuration (i.e., the affinity column was in fluid communication with the HPLC) to an off-line configuration with the HPLC (i.e., the affinity column was not in fluid communication with the HPLC). By means of a model LC10AD high pressure liquid pump (Shimadzu, Columbia, Md.), sample was transferred from the sample injector through the affinity column. Peaks were detected with a model 1100 UV-VIS detector (Agilent Technologies, Wilmington, Del.) at 361 nm.

A binding buffer comprising (0.01 M Na₂PO₄+0.15 M NaCl; pH 7.0) was pumped through the affinity column to equilibrate the column. A total of 10 column volumes of binding buffer was used.

A test sample containing vitamin B₁₂ was prepared as follows. A solid material containing approximately 1 mg/g vitamin B₁₂ and weighing 0.1 g of test sample was dissolved in 100 ml of the binding buffer to form a mixture. The mixture was filtered using a 0.22 μm filter.

Before loading the test sample, the following steps were taken:

(1) the RP-HPLC was programmed as specified below;

(2) all buffer solutions were degassed and filtered using a 0.45 μm filter;

(3) the pump tubing was filled with the appropriate buffer solutions before connecting the affinity column and analytical column(s) to the apparatus to prevent air from getting into the column(s);

(4) end-caps were removed from the columns, and the columns were connected to the apparatus;

(5) each column was equilibrated with at least 10 column volumes of binding buffer or until no signal was detected in the effluent; and

(6) test sample was loaded into a sample loop.

The following chromographic conditions were used:

| | |
|---|---|
| Column 1 | anti-vitamin B₁₂ immunoaffinity column |
| Column 2 | GENESIS ® C18, 4 μm, 4.6 × 250 mm |
| Flow rate | 1 ml/min |
| Detection | UV-VIS at 361 nm |
| Binding buffer | 0.01 M phosphate + 0.15 M NaCl, pH 7.0 |
| Elution buffer | 0.01 M HCl + 0.15 M NaCl |
| Mobile phase | 30% v/v methanol/70% v/v 0.01 M HCl + |
| | 0.15 M NaCl |

The following time periods were programmed into the apparatus settings:

| Time | Column | Column | % Binding | % Elution | % Mobile |
|---|---|---|---|---|---|
| (min) | 1 | 2 | buffer | buffer | phase |
| 0-10 | On-line | Off-line | 100 | 0 | 0 |
| 10.1-20 | On-line | On-line | 0 | 100 | 0 |
| 20.1-35.0 | Off-line | On-line | 0 | 0 | 100 |
| 35.1 | On-line | On-line | 100 | 0 | 0 |

### EXAMPLE 4

### Preparation of a Rigid Support Comprising Monoclonal Anti-Aflatoxin B1 Antibody

An exemplary rigid support was prepared as follows. 1.5 g of coupling buffer and 250 μg of monoclonal anti-aflatoxin B1 antibody (Product No. A9555 commercially available from Sigma-Aldrich (St. Louis, Mo.)) having a concentration of approximately 7.6 mg antibody per milliliter) were added to a 10 ml round bottom flask. 160 mg of NaCNBH₃ and 1 gram of Intermediate E from Example 1 were added to the flask and mixed on a shaker for 4 hours. The sample was filtered and washed 4 times with 10 ml of coupling buffer. Then, 1.5 g of coupling buffer, 160 mg of NaCNBH₃ and 50 mg of ethanolamine were added to the 10 ml round bottom flask, and then mixed on a shaker for 4 hours. The sample was filtered and washed 4 times with 10 ml of coupling buffer. The resulting rigid support material was placed in PBS buffer containing 0.02% sodium azide and stored at 4° C.

### EXAMPLE 5

### Preparation of an Affinity Column for Detecting Aflatoxin B1

An exemplary affinity column was prepared by packing a 4.6×50 mm I.D. affinity column with the rigid support material produced in Example 4. The packed column was then filled with 20 mM phosphate buffer solution, pH 7.4, containing 0.02 wt % sodium azide. The resulting exemplary affinity column was stored at a temperature of 4° C.

### EXAMPLE 6

### Analysis of an Aflatoxin-Containing Composition

The exemplary affinity column of Example 5 was coupled to an apparatus similar to exemplary apparatus 40 as shown in FIG. 3. The apparatus comprised a High Performance Liquid Chromatograph (HPLC) (model 1100 series, Agilent Technologies, Wilmington, Del.) equipped with a post-column model Cobra cell (Lamers & Pleuger's, Hertogenbosch, NL). Injection was performed with a model RHEODYNE 7725i sample injector (VWR International Ltd, Dorset, UK) equipped with a 500 μl sample loop. By means of a model RHEODYNE 7725 sample injector, it was possible to switch the affinity column on- and off-line with the HPLC. By means of a model LC10AD high pressure liquid pump (Shimadzu, Columbia, Md.), sample was transferred from the sample injector through the affinity column. Peaks were detected with a model 100 TV-VIS detector (Agilent Technologies, Wilmington, Del.) at 365 nm and a model 1046A programmable fluorescence detector (Agilent Technologies, Wilmington, Del.) at 365/430 nm.

A binding buffer comprising (0.01 M Na₂PO₄+0.15 M NaCl; pH 7.0) was pumped through the affinity column to equilibrate the column. A total of 10 column volumes of binding buffer was used.

Test samples containing Aflatoxin B1, B2, G1 and G2 were prepared as follows. A solid material containing 10 to 100 ng/g Aflatoxin B1, B2; G1 and G2 and weighing 25 g was suspended in 100 ml of acetonitrile. The samples were ultrasonically extracted for 15 minutes. The liquid fraction was centrifuged for 10 minutes at 6000 rpm. 100 μl of the supernatant was diluted with 900 μl of binding buffer.

Before loading the test sample, the following steps were taken:

(1) the RP-HPLC was programmed as specified below;

(2) all buffer solutions were degassed and filtered using a 0.45 μm filter;

(3) the pump tubing was filled with the appropriate buffer solutions before connecting the affinity column and analytical column(s) to the apparatus to prevent air from getting into the column(s);

(4) end-caps were removed from the columns, and the columns were connected to the apparatus;

(5) each column was equilibrated with at least 10 column volumes of binding buffer or until no signal was detected in the effluent; and

(6) test sample was loaded into a sample loop.

The following chromographic conditions were used:

| | |
|---|---|
| Column 1 | anti-aflatoxin B1 immunoaffinity column |
| Column 2 | GENESIS ® CIS, 4 μm, 4.6 × 250 mm |
| Detection 1 | UV-VIS at 365 nm |
| Detection 2 | Fluorescence detection (365/430 nm) |
| Binding buffer | 0.01 M phosphate + 0.15 M NaCl, pH 7.0 |
| Flow rate | 0.5 ml/min |
| Elution buffer | 20% v/v acetonitrile in water |
| Mobile phase | 600 ml methanol + 80 ml acetonitrile + 200 μl of |
| | concentrated nitric acid + 50 mg of potassium |
| | bromide and adjusted to 1000 ml with water |
| Flow rate | 0.8 ml/min |

The following time periods were programmed into the apparatus settings:

| Time | Column | Column | % Binding | % Elution | % Mobile |
|---|---|---|---|---|---|
| (min) | 1 | 2 | buffer | buffer | phase |
| 0-5 | On-line | Off-line | 100 | 0 | 0 |
| 5-10 | On-line | Off-line | 100 | 0 | 0 |
| 10-14.5 | On-line | On-line | 0 | 100 | 0 |
| 14.6-40 | Off-line | On-line | 0 | 0 | 100 |
| 40.1-50.1 | On-line | On-line | 100 | 0 | 0 |

### EXAMPLE 7

### Preparation of a Rigid Support Comprising Recombinant Human Estrogen Receptor (hER) as the Active Ligand

An exemplary rigid support was prepared as follows. 1.5 g of coupling buffer and 50 μg of recombinant human estrogen receptor (Product No. AB RP-310 commercially available from 10P's (Breda, NL)) were added to a 10 ml round bottom flask. 160 mg of NaCNBH₃ and 1 gram of Intermediate E from Example 1 were added to the flask and mixed on a shaker for 4 hours. The sample was filtered and washed 4 times with 10 ml of coupling buffer. Then, 1.5 g of coupling buffer, 160 mg of NaCNBH₃ and 50 mg of ethanolamine were added to the 10 ml round bottom flask, and then mixed on a shaker for 4 hours. The sample was filtered and washed 4 times with 10 ml of coupling buffer. The resulting rigid support material was placed in PBS buffer containing 0.02% sodium azide and stored at 4° C.

### EXAMPLE 8

### Preparation of an Affinity Column for Detecting an Endocrine Disrupter

An exemplary affinity column was prepared by packing a 4.6 mm×50 mm I.D. affinity column with the rigid support material produced in Example 7. The packed column was then filled with 20 mM phosphate buffer solution, pH 7.4, containing 0.02 wt % sodium azide. The resulting exemplary affinity column was stored at a temperature of 4° C.

### EXAMPLE 9

### Analysis of an Endocrine Disrupter-Containing Composition

The exemplary affinity column of Example 8 was coupled to an apparatus similar to the apparatus used in Example 3. A binding buffer comprising (0.01 M Na₂PO₄+0.15 M NaCl; pH 7.0) was pumped through the affinity column to equilibrate the column. A total of 10 column volumes of binding buffer was used.

A test sample containing a mixture of 17-β-estradiol, 17-α-estradiol, 17-α-ethynylestradiol, estrone, bisphenol A, nonylphenol and butylbenzyl phthalate was prepared as follows. Stock solutions containing from 250 to 1000 mg/l of 17-β-estradiol, 17-α-estradiol, 17-α-ethynylestradiol, estrone, bisphenol A, nonylphenol and butylbenzyl phthalate were diluted by a factor of 1000 in 100 ml of the binding buffer to form a mixture

Before loading the test sample, the following steps were taken:

(1) the RP-HPLC was programmed as specified below;

(2) all buffer solutions were degassed and filtered using a 0.45 μm filter;

(3) the pump tubing was filled with the appropriate buffer solutions before connecting the affinity column and analytical column(s) to the apparatus to prevent air from getting into the column(s);

(4) end-caps were removed from the columns, and the columns were connected to the apparatus;

(5) each column was equilibrated with at least 10 column volumes of binding buffer or until no signal was detected in the effluent; and

(6) test sample was loaded into a sample loop.

The following chromographic conditions were used:

| | |
|---|---|
| Column 1 | recombinant human estrogen receptor |
| | (hER) affinity column |
| Column 2 | GENESIS ® C18, 4 μm, 4.6 × 250 mm |
| Flow rate | 0.8 ml/min |
| Detection | UV-VIS at 230 nm |
| Binding buffer | 0.01 M phosphate + 0.15 M NaCl, pH 7.0 |
| Elution buffer | 25% v/v 6 M potassiumthiocyanate, 50% v/v binding |
| | buffer, 25% v/v methanol |
| Mobile phase A | 0.01 N HCl in water |
| Mobile phase B | Acetonitrile |

The following time periods were programmed into the apparatus settings:

| Time | Column | Column | % Binding | % Elution | % Mobile | % Mobile |
|---|---|---|---|---|---|---|
| (min) | 1 | 2 | buffer | buffer | phase A | phase B |
| 0-5 | On-line | Off-line | 100 | 0 | 0 | 0 |
| 5.1-10 | On-line | On-line | 0 | 100 | 0 | 0 |
| 10.1-20.1 | Off-line | On-line | 0 | 0 | 100 | 0 |
| 20.1-35.0 | Off-line | On-line | 0 | 0 | 65 | 45 |
| 50.0 | Off-line | On-line | 0 | 0 | 0 | 100 |
| 50.1-55 | Off-line | On-line | 0 | 0 | 0 | 100 |
| 55.1-65.1 | On-line | On-line | 100 | 0 | | 0 |

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. An apparatus comprising an affinity column in fluid communication with an analytical column, wherein the affinity column contains a rigid support capable of withstanding a column pressure of up to about 200 bar, said rigid support having one or more ligands bonded thereto, said one or more ligands capable of selectively bonding to one or more analytes within a given sample solution.

2. (canceled)

3. The apparatus of claim 2, wherein each inorganic particle comprises (i) an inorganic substrate and (ii) a modified substrate surface that reduces non-specific binding of unwanted materials to the inorganic substrate.

4. The apparatus of claim 3, wherein the modified substrate surface comprises one or more R₁₀ groups attached to the inorganic substrate, wherein each R₁₀ group is independently selected from the group consisting of —CH₂OH, —CH(OH)₂, —CH(OH)CH₃, —CH₂CH₂OH, —C(OH)₂CH₃, —CH₂CH(OH)₂ and —CH(OH)CH₂(OH).

5. (canceled)

6. The apparatus of claim 1, wherein the one or more ligands comprise a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof.

7-15. (canceled)

16. A rigid support suitable for use in an affinity column, said rigid support comprising a plurality of inorganic particles, wherein each particle comprises:
an inorganic substrate;
a modified substrate surface that reduces non-specific binding of non-analyte materials and ligand-specific analyte materials to the inorganic substrate; and
one or more ligands bonded to the inorganic substrate, said one or more ligands comprising a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof.

17-29. (canceled)

30. An affinity column comprising the rigid support of claim 16.

31. An apparatus comprising an affinity column in fluid communication with an analytical column, wherein the affinity column comprising the affinity column of claim 30.

32. An affinity column comprising:
a column structure having a column volume; and
a rigid support positioned in the column volume of the column structure, said rigid support comprising a plurality of inorganic particles, wherein each particle comprises:
an inorganic substrate;
a modified substrate surface that reduces non-specific binding of non-analyte materials and ligand-specific analyte materials to the inorganic substrate; and
one or more ligands bonded to the inorganic substrate,
said one or more ligands comprising a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof.

33-38. (canceled)

39. The affinity column of claim 32, wherein the modified substrate surface comprises reactive sites, wherein from about 50% to about 99% of the reactive sites are covered with R groups that are less reactive than any functional groups on the substrate surface prior to modification, and from about 50% to about 1% of the reactive sites are covered with the one or more ligands or optional linkers.

40. The affinity column of claim 39, wherein from about 70% to about 95% of the reactive sites are covered with R groups that are less reactive than any functional groups on the substrate surface prior to modification, and from about 30% to about 5% of the reactive sites are covered with the one or more ligands or optional linkers.

41-45. (canceled)

46. An apparatus comprising an affinity column in fluid communication with an analytical column, wherein the affinity column comprising the affinity column of claim 32.

47. A method of analyzing a test sample, said method comprising the steps of:
bringing the test sample into contact with the apparatus of claim 1.

48. A method of analyzing an eluent sample, said method comprising the steps of:
transferring the eluent sample from an affinity column to an analytical column, wherein the affinity column is in fluid communication with the analytical column, and
analyzing contents of the analytical column to determine a presence of one or more analytes in the eluent sample.

49. The method of claim 48, wherein the eluent sample comprises one or more analytes in a solvent, said one or more analytes being selected from the group consisting of aflatoxin B1, aflatoxin G1, aflatoxin Q1, aflatoxin B2, aflatoxin G2, Bisphenol A, 2,4-dichlorophenoxy acetic acid, 2,4,5-trichlorophenoxy acetic acid, 4-chloro-2-methyl acetic acid, 4-(2,4-dichlorophenoxy)butyric acid, estrone, 17-β-estradiol, 17-α-ethynylestradiol, lactoferrin, testosterone, nortestosterone, metobromuron, cinosulfuron, triasulfuron and prosulfuron, vinclozolin, folic acid, vitamin B₁₂ (cyanocobalamine), fenitrothion, chlorpyrifos, pirimifos, adrenalin, noradrenalin, dopamine, a compound having estrogenic activity, or combinations thereof.

50. The method of claim 49, wherein the eluent sample comprises a detectable amount of at least one mycotoxin.

51-53. (canceled)

54. The method of claim 48, wherein the transferring step comprises applying fluid pressure on the eluent sample to transport the eluent sample from the affinity column to the analytical column.

55. (canceled)

56. The method of claim 48, wherein the analyzing step comprises detecting the presence of one or more analytes in the eluent sample, separating the one or more analytes from one another, quantifying one or more analytes in the eluent sample, or any combination thereof.

57. The method of claim 56, wherein the analyzing step comprises subjecting the eluent sample to Reversed Phase High Performance Liquid Chromatography (RP-HPLC), fluorescence detection, or both.

58. The method of claim 48, wherein the affinity column contains a rigid support capable of withstanding a column pressure of up to about 300 bar, said rigid support having one or more ligands bonded thereto, said one or more ligands being capable of selectively bonding to one or more analytes within a test sample.

59-71. (canceled)

72. The method of claim 48, further comprising the steps of:
introducing a test sample into an affinity column containing a rigid support capable of withstanding a column pressure of up to about 200 bar, said rigid support having one or more ligands bonded thereto, said one or more ligands being capable of selectively bonding to one or more analytes;
allowing the test sample to come into contact with the rigid support and ligands thereon;
rinsing the rigid support to wash away any test sample components other than the one or more analytes;
introducing an eluent solution into the affinity column so that the eluent solution comes into contact with the one or more analytes bound to the ligands on the rigid support; and
allowing the eluent solution to remain in contact with the rigid support for period of time so as to form the eluent sample.

73. The method of claim 72, wherein the period of time ranges from about 5 minutes to about 15 minutes.

74. The method of claim 72, further comprising the steps of:
flushing the affinity column with a first buffer solution; and
introducing a second test sample into the affinity column.

75. A method of analyzing an eluent sample that potentially contains at least one mycotoxin, said method comprising the steps of:
transferring the eluent sample from an affinity column to an analytical column, wherein the affinity column is in fluid communication with the analytical column, and
analyzing contents of the analytical column to determine a presence of least one mycotoxin in the eluent sample.

76. The method of claim 75, wherein the eluent sample comprises a detectable amount of aflatoxin B1, aflatoxin G1, aflatoxin Q1, aflatoxin B2, aflatoxin G2, or a combination thereof.

77. A method of analyzing an eluent sample that potentially contains folic acid, vitamin B₁₂ (cyanocobalamine), or a combination thereof, said method comprising the steps of:
transferring the eluent sample from an affinity column to an analytical column, wherein the affinity column is in fluid communication with the analytical column, and
analyzing contents of the analytical column to determine a presence of folic acid, vitamin B₁₂ (cyanocobalamine), or both in the eluent sample.

78. A method of analyzing a test sample that potentially contains at least one compound having estrogen activity, said method comprising the steps of:
introducing the test sample into an affinity column containing a rigid support having one or more ligands bonded thereto, said one or more ligands being capable of selectively bonding to one or more compounds having estrogen activity.

79. The method of claim 78, wherein the, one or more ligands comprise a native human estrogen receptor, a recombinant human estrogen receptor (hER) or derivative thereof, a recombinant protein mimicking a biologically active part of an estrogen receptor or derivative thereof, or any other ligand that selectively recognises a compound having biological activity as an endocrine disrupter.

80. (canceled)

81. The method of claim 78, further comprising the steps of:
allowing the test sample to come into contact with the rigid support and ligands thereon;
rinsing the rigid support to wash away any test sample components that do not exhibit estrogen activity;
introducing an eluent solution into the affinity column so that the eluent solution comes into contact with the one or more compounds having estrogen activity bound to the ligands on the rigid support; and
allowing the eluent solution to remain in contact with the rigid support for a period of time so as to form a eluent sample containing the compounds having estrogen activity; and
analyzing contents of the analytical column to determine a presence of one or more compounds having estrogen activity in the eluent sample.

82. (canceled)

83. A method of analyzing a test sample that potentially contains at least one analyte, said method comprising the steps of:
introducing the test sample into an affinity column containing a rigid support, said rigid support comprising a plurality of inorganic particles, wherein each particle comprises:
an inorganic substrate;
a modified substrate surface that reduces non-specific binding of non-analyte materials and ligand-specific analyte materials to the inorganic substrate; and
one or more ligands bonded to the inorganic substrate, said one or more ligands comprising a monoclonal anti-aflatoxin B1 antibody, a monoclonal anti-aflatoxin G1 antibody, a monoclonal anti-aflatoxin Q1 antibody, a monoclonal anti-aflatoxin B2 antibody, a monoclonal anti-aflatoxin G2 antibody, a monoclonal anti-Bisphenol A antibody, a monoclonal anti-2,4-dichlorophenoxy acetic acid antibody, a monoclonal anti-2,4,5-trichlorophenoxy acetic acid antibody, a monoclonal anti-4-chloro-2-methyl acetic acid antibody, a monoclonal anti-4-(2,4-dichlorophenoxy)butyric acid antibody, a monoclonal anti-estrone antibody, a monoclonal anti-17-β-estradiol antibody, a monoclonal anti-17-α-ethynylestradiol antibody, a monoclonal anti-lactoferrin antibody, a monoclonal anti-testosterone antibody, a monoclonal anti-nortestosterone antibody, a monoclonal anti-phenylurea antibody, a monoclonal anti-vinclozolin antibody, a monoclonal anti-folic acid antibody, a monoclonal anti-vitamin B₁₂ (cyanocobalamine) antibody, a monoclonal anti-fenitrothion antibody, a monoclonal anti-chlorpyrifos antibody, a monoclonal anti-pirimifos antibody, an anti-catechol amine antibody, an recombinant human estrogen receptor (hER), and combinations thereof;
allowing the test sample to come into contact with the rigid support and ligands thereon;
rinsing the rigid support to wash away any test sample components that do not bond to the ligands;
introducing an eluent solution into the affinity column so that the eluent solution comes into contact with one or more analytes bound to the ligands on the rigid support; and
allowing the eluent solution to remain in contact with the rigid support for a period of time so as to form an eluent sample potentially containing one or more analytes; and
analyzing contents of the analytical column to determine a presence of one or more analytes in the test sample.

84-93. (canceled)

94. A method of making a rigid support material comprising an inorganic substrate, said method comprising the following steps:
(1) attaching R groups to at least a first portion of the surface of the inorganic substrate, wherein the R groups have a reactivity less than any functional groups on a surface of the inorganic substrate prior to the attaching step;
(2) attaching one or more linkers to at least a second portion of the surface of the inorganic substrate, wherein the one or more linkers comprise an aldehyde functional group; and
(3) selectively bonding one or more ligands to the one or more linkers.

95. The method of claim 94, wherein step (2) is performed prior to step (1).

96-98. (canceled)
